(19) United States
(12) Patent Application Publication
(10) Pub. No.: US 2004/0171071 A1
(43) Pub. Date: Sep. 2, 2004

(54) Method for separating and/or detecting and/or identifying and/or quantifying prion proteins

(76) Inventor: Elie Stefas, La Grande-Motte (FR)

Correspondence Address: YOUNG & THOMPSON, 745 SOUTH 23RD STREET 2ND FLOOR, ARLINGTON, VA, 22202

(21) Appl. No.: 10/482,378

(22) PCT Filed: Jul. 2, 2002
(86) PCT No.: PCT/FR02/02292

(30) Foreign Application Priority Data
Jul. 3, 2001 (FR) 01/08797

(51) Int. Cl. G01N 33/53

(52) U.S. Cl. 435/7.1

(57) ABSTRACT

The invention concerns a method for separating and/or detecting and/or identifying and/or quantifying in a biological material at least a prion protein (PrP), characterised in that it comprises a step which consists in separating and/or detecting and/or identifying and/or quantifying a (PrP/β2GPI) complex consisting of at least a prion protein bound to at least a form of β2GPI.

The present invention relates to a method for separating and/or detecting and/or identifying and/or quantifying prion proteins, responsible for neurodegenerative diseases, in various biological materials.

Prions belong to “unconventional transmissible agents” (UTAs) and are implicated in diseases encountered in humans and animals. They are agents responsible for neurodegenerative diseases grouped together under the name transmissible subacute spongiform encephalopathies (TSSEs). These prion-related encephalopathies include, in animals, scrapie in sheep and goats, bovine encephalopathy or mad cow disease, chronic wasting disease in wild ruminants, and mink and cat encephalopathies and, in humans, Creutzfeld-Jacob disease (CJD), Gerstmann-Straüssler-Scheinker syndrome (GSS), kuru and fatal familial insomnia (Les virus transmissibles de la mére á l'enfant [Viruses transmissible from mother to child], Ed. John Libbey Eurotext, 1999, Ermias D. Belay, Annu. Rev. Microbiol., 1999, 53: 283-314).

These always fatal diseases pose a serious public health problem, in particular due to the difficulties encountered in the early identification and detection of these agents. There is therefore a great demand with respect to reliable methods for the diagnosis and for effective therapeutic measurements of these unconventional transmissible agents.

These diseases appear to be caused by the post-translational and conformational transition of a normal cellular prion protein, hereinafter referred to as PrPC, into an abnormal pathogenic form, hereinafter referred to as PrP^{SC} (Cohen F. E. and Prusiner S. B., Annu. Rev. Biochem., 1998, 67: 793-819).

The normal cellular form of the prion protein is a cell surface glycoprotein which is highly conserved and expressed by a broad spectrum of cells, in particular by neuronal cells. Its presence is essential for the disease to be able to occur. In transmissible spongiform encephalopathies, this molecule is converted into a form which is modified from a conformational point of view and which exhibits partial resistance to proteolysis. Thus, in the brain of animals or humans exhibiting TSSEs, an accumulation of abnormal PrP^{SC} is observed in the form of fibrils and, in certain cases, in the form of amyloid deposits in the cells. This abnormal PrP^{SC} has a molecular weight of between 33 and 35 kDa before proteolysis and a molecular weight of between 27 and 30 kDa after the action of proteinase K: this resistance to proteinase K makes it possible to differentiate PrP^{SC} from PrP^{C}, which is destroyed by the action of said proteinase. Biophysical studies have also demonstrated that PrP^{C} contains a high number of α-helices (42%) and very few β-sheets, whereas, on the contrary, the PrP^{SC} form contains fewer α-helices (30%) and a high number of β-sheets (43%) and has a tendency to polymerize in the form of amyloid fibrils (Cohen F. E. and Prusiner S. B., Annu. Rev. Biochem., 1998, 67: 793-819). The prion protein, generally referred to as PrP, is also characterized by its affinity for polysulfated polyanions such as heparin sulfate and dermatan sulfate (Brimacombe B. et al., Biochem. J., 1999, 342: 605-613).

It is known that β2-glycoproteins I, hereinafter abbreviated as β2GPI, is a plasma glycoprotein the sequence of which has in particular been mentioned in articles by J. Lozier et al., Proc. Natl. Acad. Sci. USA, Vol. 81, pages 3640-3644, July 1984 and by T. Kristensen et al., FEBS Letters, Vol. 289, 1991, pages 183-186. β2GPI is also called Apolipoprotein H (APOH). It has been noted that this protein exhibits a structural polymorphism: the name β2GPI will hereinafter be considered as generic for all the forms.

This structural polymorphism is under genetic control, as was in particular indicated in the article by D K. Sanghera et al., Hum. Genet., Vol. 100, 1997, pages 57-62. It is due to the presence of four alleles: three common alleles (APOH*1, APOH*2 and APOH*3) and one rare allele (APOH*4). The APOH*3 allele has subsequently been subtyped as APOH*3^{W} and APOH*3^{D} on the basis of its reactivity with a monoclonal antibody 3D11. This polymorphism is due to several substitutions in the DNA region encoding APOH, such as Ser88Asn (D K. Sanghera et al., Hum. Genet., Vol. 100, 1997, pages 57-62), Val247Leu (A. Steinkasserer et al., Hum. Genet., Vol. 91, 1993, pages 401-402), Cys306Gly (D K. Sanghera et al., Hum. Mol. Genet., Vol. 6, 1997, pages 311-316), and Trp316Ser (D K. Sanghera et al., Hum. Genet., Vol. 100, 1997, pages 57-62 and DK. Sanghera et al., Hum. Mol. Genet., Vol. 6, 1997, pages 311-316). The mutations Ser88Asn and Trp316Ser correspond to the APOH*1 and APOH*3^{W} alleles respectively. The β2′GPI form described in FR-2 701 260 B1 results from the mutation Thr318Ser.

β2GPI is known to be a glycoprotein having high affinity for anionic phospholipids such as cardiolipin (H. Wurm, Int. J. Biochem., Vol. 16, 1984, pages 511-515).

International application WO 94/18569 has indicated that viral compounds bind specifically to a form of β2GPI, namely that described in French patent application 2 701 263, whether this form of β2GPI is in the pure state or in a protein composition containing it; this form of β2GPI is isolated from the residue attached to the affinity chromatography column(s) used in the method for purifying blood plasma albumin described in FR-A-2 690 444; it has a molecular weight of 50 000±3 000 daltons.

The hypothesis that the binding of β2GPI to viral compounds might involve the phospholipids present on these compounds has been put forward (H. Mehdi et al., J. Virol., Vol. 68 (4), 1994, pages 2415-2424, AR. Neurath et al., Virology, Vol. 204 (1), 1994, pages 475-477, E. Stefas et al., AIDS Res. Hum. Retr., Vol. 13 (1), 1997, pages 97-104, E. Stefas et al., Hepatology, Vol. 33 (1), 2001, pages 207-217). It has also been described that β2GPI serves as a cofactor for the binding of anti-phospholipid antibodies to anionic phospholipids (M. Galli et al., Lancet, Vol. 335 (8705), 1990, pages 1544-1547, H P. McNeil et al., Proc. Natl. Acad. Sci. USA, Vol. 87 (11), 1990, pages 4120-4124). In the literature, the hypothesis that the lysine residues of β2GPI are responsible for the binding of the latter to anionic phospholipids has also been put forward (Steinkasserer A. et al., Biochemical Journal, 1991, 277 (Pt 2): 387-391); this hypothesis was supported by the fact that modification of said lysine residues by carbamylation abolished its binding to phospholipids (Arvieux J. et al., Thrombosis and Haemostasis, 70 (2), 1993: 336-341, Kertesz Z. et al., Biochemical Journal, 310, 1995: 315-321).

Consequently, it appears that the binding between an infectious compound and β2GPI involves protein-phospholipid interactions, and also other types of binding, in particular based on protein-protein interactions.

It is clearly apparent from the data in the medical literature that there is an urgent need to develop methods for isolating, identifying and diagnosing pathogenic agents such as PrP^{SC} abnormal prion proteins which cause known diseases and emerging diseases, and methods for isolating and identifying PrP^{C} normal prion proteins from which the PrP^{SC} proteins derive. These methods will ultimately make it possible to understand more clearly these pathogenic agents and also the mechanisms which they used to infect living organisms.

Consequently, the aim of the present invention is to provide:

a method for separating a prion protein from a biological material, and/or

a method for isolating and purifying a prion protein, and/or

a method for detecting a prion protein in a biological material, and/or

a method for identifying a prion protein in a biological material, and/or

a method for quantifying a prion protein in a biological material.

According to the invention, it has been noted, surprisingly and unexpectedly, that PrP prion proteins can be separated and/or detected and/or identified and/or quantified by virtue of their binding to the various forms of β2GPI. The term “binding” indicates that these PrPs are physically connected to, and interact with, the various forms of β2GPI. This binding can be demonstrated by any method or assay known on the subject, such as assays using biotin and avidin or streptavidin, of the immunoenzyme type, such as ELISA or Immunoblotting, of the radioimmunoassay type, such as RIA, competition assays, agglutination assays, immunoprecipitation assays, chromatography assays, etc. In general, the term “complex” will be used here for a direct or indirect association between at least one PrP, which may be normal or abnormal, and at least one form of β2GPI; these complexes will in general be referred to as “PrP/β2GPI”.

In the present patent application, the term “PrP” means, generically, both the protein compounds constituting a PrP and PrP-type particles. PrP-type particles are either complete or incomplete PrPs, or parts of PrP, or assemblies containing compounds constituting PrP, which exhibit certain properties of PrPs or of PrP compounds, in particular those to be detected by certain antibodies specific for PrP compounds.

According to the present invention, the term “biological material” means a biological tissue, or a preparation or an extract derived from the biological tissue, which may be liquid or solid, or a natural medium, which may be liquid or solid, liable to contain or carry a PrP within the meaning defined above. The material may thus be a mixture of at least two materials as defined above. Such a biological material may therefore in particular be either prepared from tissues, from organs, from stools or from biological fluids from a patient suffering from an infection due to a PrP^{SC}, or obtained from “in vitro” cultures; such a biological material may also be a serum, plasma, urine, cerebrospinal fluid, synovial fluid, peritoneal fluid, pleural fluid, seminal fluid, saliva, gastric secretions, mucus, ascites fluid, or the like.

A subject of the present invention is therefore a method for separating and/or detecting and/or identifying and/or quantifying, in a biological material, at least one prion protein (PrP), characterized in that it comprises a step of separating and/or detecting and/or identifying and/or quantifying a complex (PrP/β2GPI) made up of at least one prion protein bound to at least one form of β2-glycoprotein I (β2GPI).

According to a particularity, the method comprises a step of separating and/or detecting and/or identifying and/or quantifying a complex (PrP^{SC}/β2GPI) made up of at least one abnormal prion protein (PrP^{SC}) bound to at least one form of β2GPI, said method constituting a method for separating and/or detecting and/or identifying and/or quantifying, in a biological material, at least one abnormal prion protein. In particular, to form a complex (PrP^{SC}/β2GPI), use may be made of at least one abnormal prion protein PrP^{SC} originating from scrapie in sheep or goats, bovine encephalopathy, chronic wasting disease in wild ruminants, mink or cat encephalopathies, Creutzfeld-Jacob disease (CJD), Gerstmann-Straüssler-Scheinker syndrome (GSS), kuru or fatal familial insomnia. In addition, to form a complex (PrP/β2GPI) use may be made of at least one β2GPI of human origin or of animal origin, a recombinant β2GPI, a β2GPI obtained by chemical synthesis or a modified form of β2GPI.

Advantageously, before the step of separating and/or detecting and/or identifying and/or quantifying the complex (PrP^{SC}/β2GPI), the biological material is subjected to the action of detergents and/or of enzymes, in particular to the action of proteinase K.

According to a first embodiment of the invention, a step of attaching PrP contained in a biological material to at least one form of β2GPI intentionally added to said biological material is carried out so as to form said complex, followed by a step of separating and/or detecting and/or identifying and/or quantifying the complex (PrP/β2GPI).

According to one embodiment, the following are carried out: a step of attaching at least one form of β2GPI or said PrP(s) to a support, before or after the step of attaching said PrP(s) to said form(s) of β2GPI so as to form said complex, a separation step consisting in separating the biological material from the support to which the complex is attached, and a step of detection and/or identification and/or quantification consisting, after said separation step, in detecting and/or identifying and/or quantifying the complex attached to the support, via its component which is not bound to the support. Advantageously, a solid support is used as support.

According to an implementation variant, the attachment to the support is carried out by virtue of a compound which binds to one of the PrP or β2GPI components of the complex, said step of detection and/or identification and/or quantification consisting in detecting and/or identifying and/or quantifying the complex via its component which is not bound to the support. Advantageously, the compound which binds to the β2GPI or to the PrP is an antibody which recognizes respectively the β2GPI or the PrP, or else another protein, a biological compound, a chemical compound or a detergent which attaches to the PrP or to the β2GPI.

The step of attaching at least one form of β2GPI or the PrP(s) to a support can be carried out by reacting reactive groups of the form(s) of β2GPI or of the PrP(s) with reactive sites of the support, said form(s) being dissolved in a buffer having a pH of between 2.5 and 10.5, preferably between 5.5 and 7.5, so as to obtain a solution having a concentration of between 0.01 and 100 g/l of form(s) of β2GPI or of PrP, the support being kept in contact with the solution at a temperature of between 0° and 40° C. for an incubation period of between 10 seconds and 24 hours, and then the separation of the support and the solution is carried out by washing the support.

The step of attaching at least one PrP to at least one form of β2GPI so as to form a complex can be carried out by bringing at least one form of β2GPI into contact with the biological material liable to contain PrPs at a temperature of between 0° and 50° C., advantageously in the region of 37° C., for a period of time of between 10 seconds and 24 hours, the biological material being diluted with a buffer giving a pH of between 3.5 and 10, preferably between 5.6 and 7.6.

The detection and/or identification and/or quantification of the PrP(s) of the complex can be carried out using PrP-specific antibodies, or using methods for infecting cells or organisms susceptible to PrP infection; advantageously, the detection and/or identification and/or quantification of the PrP(s) of the complex is carried out using an antibody which specifically recognizes an antigen, preferably protein in nature, of the PrP(s).

According to another embodiment, it is also possible to carry out the detection and/or identification and/or quantification of the β2GPI of the complex using β2GPI-specific antibodies.

Advantageously, the antibody used is coupled to an enzyme label, colloidal gold, or a radioactive, fluorescent or luminescent tracer. It is possible to couple the antibody to an enzyme label for which the enzyme is brought into contact with a specific substrate able to be converted into a colored product.

When the complex is attached to the support by virtue of its β2GPI component, the separation step comprises isolating the PrP of the complex attached to the support by an affinity chromatography elution method. Advantageously, said isolation is carried out by elution of the PrP attached to the solid support using a buffer having a pH of between 2 and 10.5 and an NaCl concentration of between 0 and 5M, preferably using a 0.1 mol/liter glycine-HCl buffer having a pH of 2.5.

According to a particularity of the invention, the separation of the support and the solution is followed by a step of saturating the active sites of the support, by reacting a bovine serum albumin or casein solution on the active sites.

According to another use of the invention, the method for separating and/or detecting and/or identifying and/or quantifying at least one PrP in a biological material which naturally contains at least one form of β2GPI comprises a step of separating- and/or detecting and/or identifying and/or quantifying a complex (PrP/β2GPI) made up of at least one PrP bound to at least one form of β2GPI naturally present in said material.

The forms of β2GPI used, according to the present invention, to form the complexes (PrP/β2GPI) may, as indicated above, be of human, animal or recombinant origin or may be obtained by chemical process or may be modified forms of β2GPI.

The term “of human origin” refers to any natural form of β2GPI found in humans or obtained after culturing human cells, or to fragments (combination of some amino acids, such as polypeptides or peptides) thereof, obtained either during purification, by enzymatic cleavage(s) caused by enzymes already present in the biological fluids, or after purification, using enzymes which may or may not be specific for sites present on these natural human forms.

The term “of animal origin” refers to any natural form of β2GPI found in animals, obtained after culturing animal cells, or to fragments (combination of some amino acids, such as polypeptides or peptides) thereof, obtained either during purification, by enzymatic cleavage(s) caused by enzymes already present in the biological fluids, or after purification, using enzymes which may or may not be specific for sites present on these natural animal forms.

The term “of recombinant origin” refers either to any recombinant form of β2GPI obtained according to DNA recombination techniques as described by Maniatis (Maniatis T. et al., Molecular cloning: a laboratory manual. Cold Spring Harbor Laboratory Press, Cold Spring Harbor, N.Y., 1982), after insertion of the gene, which may or may not be modified, and genetic recombination or after modification of the gene already expressed using methods known in the field in bacteria or other cells used in the production of recombinant proteins, or else to fragments (combination of some amino acids, such as polypeptides or peptides) of such recombinant forms, obtained either during purification, by enzymatic cleavage(s) caused by enzymes already present in the biological fluids, or after purification, using enzymes which may or may not be specific for sites present on these recombinant forms.

The term “modified” refers to any natural form of β2GPI found in humans or animals, to any form of recombinant origin, to any form obtained after culturing human or animal cells, or else to fragments (combination of some amino acids, such as polypeptides or peptides) of these forms, obtained either during purification, by enzymatic cleavage(s) caused by enzymes already present in the biological fluids, or after purification, using enzymes which may or may not be specific for sites present on said forms, after all the above-mentioned forms have undergone modifications, for example by chemical process, of some of their amino acids, such as, for example, carbamylation of the lysine residues.

The term “obtained by chemical process” refers to any form of β2GPI of human, animal or recombinant origin as defined above, obtained by chemical synthesis. In particular, the polypeptides and peptides originating from said forms of β2GPI may be prepared according to any method known in the field, in particular by conventional chemical synthesis as described by Atherton and Shepard in “Solid phase peptide synthesis”, IRL Press, Oxford, 1989. It is clear that the terms “polypeptides” and “peptides” refer to a polymer of amino acids comprising fewer amino acids than the natural protein sequence but do not exclude post-translational modifications of the polypeptides and peptides, such as glycosylation, acylation, phosphorylation, modifications with fatty acids or the like. Also included in the definition are polypeptides and peptides with amino acid substitutions, mutated versions or variations in the natural sequence of these polypeptides and peptides, polypeptides and peptides with substituted bonds, polypeptides and peptides containing cystein residues connected by disulfide bridges, cystein residues without disulfide bridges, along with the other modifications known in the field.

According to the present invention, forms of β2GPI which may be used include pure β2GPI or β2GPI in the form of a protein composition containing, in particular, other glycoproteins. The form of. 2GPI may thus be obtained:

from human or animal plasma or other biological fluid, for example serum, urine, cerebrospinal fluid, using purification methods already described in the literature or in French patent 2 701 263; or

commercially; or

from supernatants of immortalized cells which express it; or

by expression of the gene which encodes it, in bacteria or other cells used in the production of recombinant proteins; or

by chemical synthesis.

The forms of β2GPI can be characterized and sequenced according to any method known in the field.

To implement the method according to the invention, it is possible either to carry out the detection and/or identification and/or quantification of PrP without prior attachment of the complex (PrP/β2GPI) to a support or to carry out the separation and/or detection and/or identification and/or quantification of PrP without attachment of said complex to a support, via an element constituting the complex; in the first case, the detection and/or identification and/or quantification is carried out in the medium in which the complex has formed, either after attachment of said medium by a physical, chemical or biochemical method, for example, to a surface, or without attachment of said medium; in the second case, the support may advantageously be a solid support, the separation consisting in separating the support to which the complex is attached, the detection and/or identification and/or quantification consisting in detecting and/or identifying and/or quantifying the complex attached to the support after having separated said support from the biological material.

The term “solid support” refers to any solid support known in the field, such as one of those described in “Current Protocols in Immunology” from Editions Coligan J., Kruisbeek A., Margulies D., Shevach E. and Strober W., Wiley Interscience, 1992. This support may, for example, be a microtitration plate of the ELISA type, a membrane, in particular a nitrocellulose membrane, a chromatography gel, beads, in particular polystyrene beads, tubes, in particular polystyrene or polypropylene tubes, or live, human or animal or bacterial or viral cells.

According to a first embodiment of the invention when the complex is attached to a support, the complex (PrP/β2GPI) is retained on the support by virtue of the β2GPI component of the complex; next, the component of the complex corresponding to the PrP is detected/identified/quantified or isolated by any suitable means. The attachment of the β2GPI component to the support can be carried out after the formation of the complex or, preferably, before the formation of the complex. If the attachment of the β2GPI component is performed before the formation of the complex, said attachment to the solid support is carried out by reacting reactive groups of the form(s) of β2GPI with reactive sites of the support according to any process known in the field, as described in “Current Protocols in Immunology” from Editions Coligan J., Kruisbeek A., Margulies D., Shevach E. and Strober W., Wiley Interscience, 1992. This reaction is preferably carried out at a temperature of between 0° and 40° C., the form(s) of β2GPI preferably being placed in a buffer having a pH of between 2.5 and 10.5, advantageously between 5.5 and 7.5. An isotonic or virtually isotonic buffer is preferably used. The buffer may be of the phosphate or acetate type. The solution obtained advantageously has a concentration of between 0.01 and 100 g/l of form(s) of β2GPI. The support is advantageously kept in contact with the buffer containing the form(s) of β2GPI at a temperature of between 0 and 40° C. and for an incubation period of between 10 seconds and 24 hours. After incubation, the buffer containing the form(s) of β2GPI which has(have) not reacted is separated from the support and washing of the support is performed, preferably with the same buffer as that which contained the form(s) of β2GPI. It may be necessary to saturate the active sites of the support which have not reacted with the form(s) of β2GPI. In this case, other active groups chosen from solutions of bovine albumin of fetal calf serum, of casein or the like are reacted on these active sites. For this purpose, use is advantageously made of a solution of bovine serum albumin, in particular a solution at 2% in the buffer used for the form(s) of β2GPI. After reaction, the support is preferably rinsed and dried.

The reaction of the solid support carrying one or more form(s) of β2GPI with the biological material is carried out according to any method known in the field, such as those described in “Current Protocols in Immunology” from Editions Coligan J., Kruisbeek A., Margulies D., Shevach E. and Strober W., Wiley Interscience, 1992. The support to which the form(s) of β2GPI is(are) attached is then brought into contact with a biological material liable to contain PrPs. The biological material is preferably diluted using a buffer which gives a pH of between 3.5 and 10, advantageously between 5.6 and 7.6. The reaction is preferably carried out at a temperature of between 0° and 50° C., advantageously in the region of 37° C., for a period of time of between 10 seconds and 24 hours. The biological material can then be separated from the support carrying the form(s) of β2GPI which has (or have) possibly attached at least one PrP. Washing is then optionally carried out with a preferably buffered solution.

The same conditions for attaching the form(s) of β2GPI to the support and for attaching the PrP(s) to the form(s) of β2GPI as those described above can be used when the attachment of the form(s) of β2GPI to the support is carried out after the formation of the complex.

When the complex is attached to the support by virtue of its β2GPI component, it is then possible to isolate the PrP. The isolation of the PrP component of the complex attached to the solid support via its β2GPI component may be carried out according to any elution method used for affinity chromatography, such as those described in “Guide to protein purification. Methods in enzymology, published by Deutscher M., Academic Press, 1990. The biological material is separated or eluted from the solid support containing the form(s) of β2GPI using a buffer having a pH of between 2 and 10.5, and having an NaCl concentration of between 0 and 5M, advantageously with a 0.1 mol/liter glycine-HCl buffer having a pH of 2.5.

The detection and/or identification and/or quantification of the PrPs attached to the form(s) of β2GPI may be carried out by any known means using detection and/or identification and/or quantification by antibodies, as described in “Current Protocols in Immunology” from Editions Coligan J., Kruisbeek A., Margulies D., Shevach E. and Strober W., Wiley Interscience, 1992. The term “antibodies” used above refers to polyclonal or monoclonal antibodies. The term monoclonal antibody” refers to a composition of antibodies consisting of a homogeneous population of antibodies; this term is not limited with regard to the species producing this antibody or with regard to its source of origin, or with regard to the manner in which it was produced. The detection and/or identification and/or quantification of the PrPs attached to the form(s) of β2GPI are preferably carried out using an antibody which specifically recognizes antigens, preferably protein in nature, of the PrPs. In a known manner, this antibody may be conjugated to an enzyme label, to colloidal gold, or to a radioactive, fluorescent or luminescent tracer. The excess antibody can be eliminated by washing. When the antibody is coupled to an enzyme label, it is then possible, in a known manner, to add a substrate specific for the enzyme conjugated to the antibody, which substrate is converted, under set conditions, into a colored product. The formation of said colored compound indicates the presence of the PrP and allows identification and also quantification thereof.

The detection and/or identification and/or quantification of the PrPs attached to the form(s) of β2GPI may be carried out by any known means using detection and/or identification and/or quantification by methods for infecting cells or organisms susceptible to PrP infection, as described in “Fields Virology”, Third Edition, Lippincott—Raven Publishers, 1996, or “Virology Methods Manual”, edited by Mahy B., Kangro H., Academic Press, 1996.

According to a second embodiment of the invention, when the complex is attached to a support, the complex PrP/β2GPI is retained on the support by virtue of the PrP component of said complex; the β2GPI component of said complex is then detected by any suitable means, advantageously using β2GPI-specific antibodies conjugated in particular to an enzyme label, to colloidal gold, or to a radioactive, fluorescent or luminescent tracer. The natural presence or the addition of detergent(s) and/or of lipid(s) may assist the attachment of these antibodies. The attachment of the PrP to the support may be carried out, before or after the formation of the complex, under the same conditions as those described above for the attachment of β2GPI to the support, and the attachment of the β2GPI to the PrP so as to form the complex may be carried out under the same conditions as those described above for attaching the PrP to the β2GPI.

In a first variant of the abovementioned two embodiments, the complex PrP/β2GPI is retained indirectly by virtue of the β2GPI component of the complex by equipping the support with a compound which binds to said β2GPI component; the PrP component of the complex is then detected as described above. The compound which binds to the β2GPI may, for example, be an antibody which recognizes the β2GPI or another protein, for example of viral origin or of prokaryotic or eukaryotic cellular origin, or a biological compound, for example a fatty acid or a lipid, or a chemical compound, for example dextran sulfate, heparin sulfate or a detergent, such as that known under the tradename “Triton X 100”.

In a second variant, the complex PrP/β2GPI is retained indirectly by virtue of the PrP component of said complex by equipping the support with a compound which binds to said PrP component of the complex; the β2GPI component of said complex is then detected and/or identified and/or quantified as described above. The compound which binds to the PrP may, for example, be an antibody which recognizes the PrP or another protein, for example of viral origin or of prokaryotic or eukaryotic cellular origin, or a biological compound, for example a fatty acid or a lipid, or a chemical compound, for example dextran sulfate, heparin sulfate or a detergent, such as that known under the tradename “Triton X 100”.

In these two variants, the indirect attachment of the form(s) of β2GPI or of the PrP(s) to the support by virtue of a compound can be carried out, before or after the formation of the complex, under conditions similar to those described in the case of a direct attachment.

According to another embodiment of the invention, use is made of the form(s) of β2GPI naturally present in a biological material. In this case, it is proposed to detect and/or identify and/or quantify PrPs when these PrPs are in an amount such that, with respect to the β2GPI naturally present in the biological material, they are mainly complexed or can mainly be complexed with at least one of the forms of β2GPI naturally present. In this case, the forms of β2GPI comprise any forms of animal or human β2GPI naturally present in the biological material. The complex naturally formed in the biological material can then be attached to a support, directly or indirectly, either via its PrP component or via its β2GPI component, the detection and/or identification and/or quantification of the complex being carried out via the portion of the complex not directly or indirectly bound to the support. This method may make it possible to possibly detect an initial state of the pathological condition, whereas the method according to the first embodiment is more suitable for studying a corresponding declared pathological state.

The description given below, by way of purely illustrative and nonlimiting examples, will make it possible to understand the invention more clearly. The examples are described with reference to the attached drawing in which:

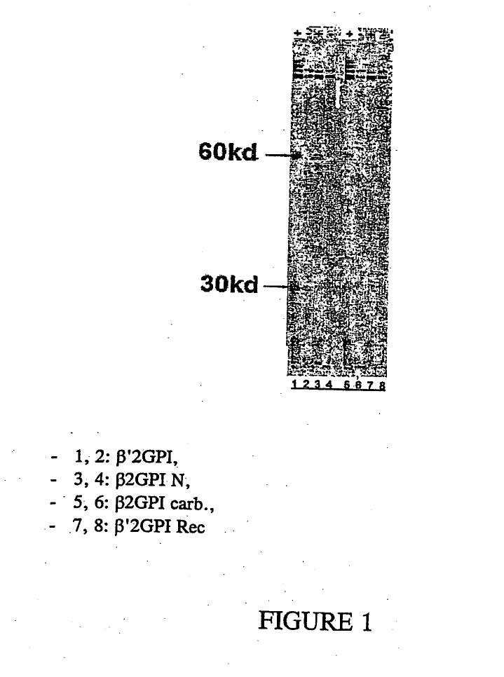

FIG. 1 represents the results obtained in example 1;

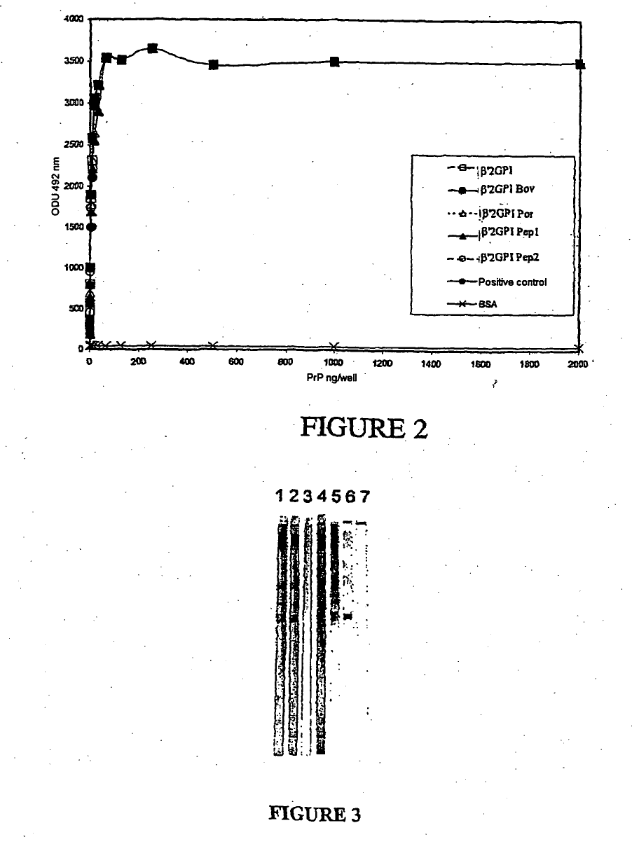

FIG. 2 represents the results obtained in example 2; and

FIG. 3 represents the results obtained in example 4.

### OBTAINING THE FORMS OF β2GPI

As starting raw material, use is made of a plasma or a serum of human or animal origin or a recombinant protein. The animal plasma or serum is either of bovine, porcine or ovine origin.

The purification of the forms of β2GPI is carried out either according to the method described in French patent 2 701 263 or according to methods already described in the literature, in particular “Gambino R, Ruiu G, Pagano G, Cassader M. Chem Phys Lipids, 1999; 103(1-2): 161-74”, “Regnault V, Arvieux J, Vallar L, Lecompte T. J Immunol Methods, 1998; 211(1-2):191-7”, “Klaerke D A, Rojkjaer R, Christensen L, Schousboe I. Biochim Biophys Acta, 1997; 1339(2): 203-16”, “Cai G, Guo Y, Shi J. Protein Expr Purif, 1996; 8(3): 341-6”, “Gambino R, Ruiu G, Cassader M, Pagano G. J Lipid Res, 1996; 37(4): 902-4”, “Williams S C, Sim R B. J Immunol Methods, 1993; 157 (1-2): 25-30”, “McNeil HP, Simpson R J, Chesterman C N, Krilis S A. Proc Natl Acad Sci USA 1990; 87(11): 4120-4”, “Lozier J, Takahaski N, Putnam F W. Proc Natl Acad Sci USA, 1984; 81(12): 3640-4”, or “Lambin P, Burstein M. Biochimie, 1982; 64(11-12): 1065-71”.

The sequence of the first 20 amino acids of the N-terminal region of the forms of β2GPI obtained after purification or after purification and fragmentation into polypeptides using proteolytic enzymes was determined by microsequencing with an “Applied Biosystems Inc, model 470” device coupled to a phenylthiohydantoin analyzer model 120 A (ABI). The sequences obtained correspond to those described in the literature and found in the databanks.

For reasons of convenience, the forms of β2GPI will hereinafter be referred to as:

β2GPI N: native β2GPI, the sequence of which corresponds to that published by “T. Kristensen et al., FEBS Letters, Vol. 289, 1991, pages 183-186”,

β′2GPI: β2GPI, the sequence of which was given in French patent 2 701 263. This form of β2GPI carries the substitution Thr318Ser;

β′2GPI carb.: β′2GPI in which the lysine residues have been modified by carbamylation with potassium cyanate at pH 5.8 for 4 hours at 37° C. according to the method described by GE Means and RE Feeney, in “Chemical modification of proteins, Holden-Day Inc, San Francisco, 1971: 215-216”;

β2GPI Bov: β2GPI purified from bovine serum;

β2GPI Por: β2GPI purified from pig plasma;

β2GPI Rec: recombinant β2GPI produced in insect cells after infection with baculovirus used as vector for the β2GPI N gene;

β2GPI Pep1: peptide corresponding to the sequence CKNEKKC of the β2GPI and obtained by chemical synthesis. The two cysteins are connected by a disulfide bridge;

β2GPI Pep2: peptide corresponding to the sequence CKNEKKC of the β2GPI and obtained by chemical synthesis. The two cysteins are free.

### EXAMPLE 1

Tissue preparations from animals infected with the bovine spongiform encephalitis (BSE) agent were prepared as described in “Maignien T. et al., Journal of General Virology, 1999, 80: 3035-3042”.

The PrP^{SC} (prion protein responsible for spongiform encephalitis transmission) was purified, according to the above reference, by centrifugation in the presence of detergents, after digestion with proteinase K. During this treatment, the PrP^{C} is destroyed by the proteinase K and the detergent(s). The purified samples were subsequently separated on a 12% polyacrylamide electrophoresis gel, in the presence of sodium dodecyl sulfate (SDS), and then transferred onto a nitrocellulose membrane (Schleicher & Schuell). The membrane was then saturated with 2% bovine albumin for 1 hour at ambient temperature and then cut up into several strips.

β′2GPI, β2GPI N, β2GPI carb. and β′2GPI Rec, coupled to alkaline phosphatase, were deposited onto these strips (assays 1, 3, 5 and 7 respectively) and, by way of comparison, onto strips obtained from tissue preparations from animals not infected with the bovine spongiform encephalitis (BSE) agent (assays 2, 4, 6 and 8 respectively).

The experiment shown by FIG. 1 was carried out as follows: the strips were rinsed three times with 50 mM acetate buffer, pH 5.6, containing 0.05% Triton X100. A milliliter containing 1 μg/ml of the various forms of β2GPI, in 50 mM acetate buffer, pH 5.6, containing 0.1% gelatine and 0.5% Triton X100, was added to each strip. After incubation for one hour at ambient temperature and with stirring, the strips were washed 6 times with PBS (phosphate buffered saline) containing 0.05% of Triton X 100. Revelation of the forms of β2GPI attached was carried out with a liquid mixture of substrate 5-bromo-4-chloro-3-indolyl phosphate/nitro blue tetrazolium (BCIP/NBT).

FIG. 1 shows that, with the various forms of β2GPI, it is possible to detect PrP^{SC} in the case of animals infected with the bovine spongiform encephalitis (BSE) agent.

### EXAMPLE 2

Recombinant bovine protein PrP was obtained from the company “Prionics”. The solid support used is a 96-well and flat-bottomed microtitration plate of the “C8 Starwell Maxisorp” type, sold by the company “NUNC”.

The experiment of attachment to a solid support to which one of the forms of β2GPI was attached was carried out as follows. The recombinant bovine PrP was used over a concentration range of 0 to 2000 ng/well. The dilution was performed using a buffer (Tris/HCl) having a Tris concentration of 0.05 mol/l and a pH of 7.6±0.05. 100 μl of solution are deposited at the bottom of each well of the plate. The plate is incubated at +37° C. for a period of 90 minutes. After this incubation, washing is carried out by introducing 300 μl of phosphate buffer into each well, which is left in contact for 2 minutes, and the buffer solution is suctioned off; this washing operation is repeated 4 times.

The revelation of the PrP attached to the various forms of β2GPI was carried out using a solution of monoclonal antibody 6H4, obtained from the company “Prionics”.

This monoclonal antibody 6H4 is specific for PrP. 100 μl of a solution of monoclonal antibody 6H4 diluted 5000-fold in phosphate buffered saline (PBS) were added per well. The plate was left to incubate at 37° C. for 60 minutes. Subsequent to this incubation, the content of the wells of the plate is suctioned off. 300 μl of phosphate buffer are introduced into each well and, after a contact period of 2 minutes, the buffer is suctioned off; this washing operation is repeated 4 times.

100 μl of a solution of rabbit antibody specific for mouse IgGs, conjugated to peroxidase, are added per well. The plate is left to incubate at 37° C. for 60 minutes. Subsequent to this incubation, the content of the wells of the plate is suctioned off. 300 μl of phosphate buffer are introduced into each well and, after a contact period of 2 minutes, the buffer is suctioned off; this washing operation is repeated 6 times.

100 μl of a solution of o-phenylenediamine, 2HCl, in a sodium citrate buffer, are added per well. This is left to incubate for 30 minutes at ambient temperature, and the reaction is then stopped by adding 50 μl of 2N H₂SO₄ to each well. The absorbance at 492 nm obtained at the end of the reaction is measured using a plate reading robot.

Table 1 and the corresponding FIG. 2 show that the recombinant PrP, the sequence of which is that of two forms of prion protein PrP^{C} and PrP^{SC}, can be detected with the various forms of β2GPI. The positive control is recombinant PrP attached to the solid support at the various concentrations given in table 1 and then revealed with the solution of monoclonal antibody 6H4. BSA (bovine serum albumin) serves as a negative control demonstrating that the binding is specific to the forms of β2GPI.

TABLE 1
Optical density values (ODU × 1000)

| PrP | | | | | | Posi- | |
|---|---|---|---|---|---|---|---|
| concen- | | | | | | tive | |
| tration | | β2GPI | β2GPI | β2GPI | β2GPI | con- | |
| ng/well | β′2GPI | Bov. | Por | Pep1 | Pep2 | trol | BSA |
| 2000 | 3500 | 3500 | 3500 | 3500 | 3500 | 3500 | 42 |
| 1000 | 3500 | 3500 | 3500 | 3500 | 3500 | 3500 | 42 |
| 500 | 3458 | 3458 | 3458 | 3458 | 3458 | 3458 | 42 |
| 250 | 3648 | 3648 | 3648 | 3648 | 3648 | 3648 | 43 |
| 125 | 3521 | 3521 | 3521 | 3521 | 3521 | 3521 | 43 |
| 62.5 | 3542 | 3542 | 3542 | 3542 | 3542 | 3542 | 42 |
| 31.25 | 3215 | 3215 | 2915 | 2904 | 3215 | 3215 | 45 |
| 15.62 | 2985 | 3060 | 2650 | 2558 | 2965 | 2965 | 42 |
| 7.812 | 2315 | 2580 | 2310 | 2214 | 2212 | 2105 | 43 |
| 3.91 | 1856 | 1896 | 1782 | 1695 | 1746 | 1500 | 44 |
| 1.95 | 798 | 1005 | 698 | 649 | 956 | 785 | 43 |
| 0.98 | 463 | 564 | 369 | 322 | 452 | 256 | 42 |
| 0.49 | 286 | 365 | 195 | 188 | 298 | 185 | 42 |

### EXAMPLE 3

Recombinant bovine protein PrP, identical to that used in example 2, was added to human serum.

The solid support used is a 96-well and flat-bottomed microtitration plate of the “C8 Starwell Maxisorp” type, sold by the company “NUNC”. Various solutions of compounds were attached to the support, namely a solution of recombinant proteins p26-HIV2 ROD recognizing the β2GPI, monoclonal antibodies recognizing the β2GPI, dextran sulfate, heparin sulfate recognizing the PrP prion protein and also β2GPI, and monoclonal antibodies recognizing the PrP prion protein. The β2GPI used in this example is β2′GPI. The serum containing the recombinant bovine PrP was diluted 50-fold. The dilution is performed using a buffer (Tris/HCl) having a Tris concentration of 0.05 mol/l and a pH of 7.6+0.05. 100 μl of solution are deposited at the bottom of each well of the plate. The plate is incubated at +37° C. for a period of 90 minutes. After this incubation, washing is carried out by introducing 300 μl of phosphate buffer into each well, which is left in contact for 2 minutes, and the buffer solution is suctioned off; this washing operation is repeated 4 times.

Revelation of the PrP component of the complex was carried out using a solution of monoclonal antibody 6H4, obtained from the company “Prionics”. Revelation of the β2GPI component of the complex was carried out using a solution of monoclonal antibody 8C3.

100 μl of a solution of monoclonal antibody 6H4 or 8C3 diluted 5000-fold in phosphate buffered saline (PBS) are added per well. The plate is left to incubate at 37° C. for 60 minutes. Subsequent to this incubation, the content of the wells of the plate is suctioned off. 300 μl of phosphate buffer are introduced into each well and, after a contact period of 2 minutes, the buffer is suctioned off: this washing operation is repeated 4 times.

100 μl of a solution of mouse monoclonal antibody specific for mouse IgGs, conjugated to peroxidase, are added per well. The plate is left to incubate at 37° C. for 60 minutes. Subsequent to this incubation, the content of the wells of the plate is suctioned off. 300 μl of phosphate buffer are introduced into each well and, after a contact period of 2 minutes, the buffer is suctioned off: this washing operation is repeated 6 times.

100 μl of a solution of o-phenylenediamine, 2HCl, in a sodium citrate buffer, are added per well. This is left to incubate for 30 minutes at ambient temperature and the reaction is then stopped by adding 50 μl of 2N H₂SO₄ to each well. The absorbance at 492 nm obtained at the end of the reaction is measured using a plate reading robot.

The mean of the absorbances obtained for each solution of compound attached to the support is given in table 2 (in optical density units multiplied by 1000). The results in table 2 demonstrate that the complex PrP/β2GPI can be detected according to the method of the present invention and, in particular, that the recombinant PrP can be detected.

TABLE 2
Optical density values (ODU × 1000)

| | Revelation of the | Revelation of the |
|---|---|---|
| Compound attached | β2GPI component | PrP component of |
| to the support | of the complex | the complex |
| Heparin sulfate | 1896 | 1756 |
| P26-HIV2 ROD | — | 1615 |
| Dextran sulfate | 1752 | 1459 |
| Anti-β2GPI⁽¹⁾ | — | 1763 |
| antibody | | |
| Anti-PrP^{SC(2)} | 1862 | — |
| antibody | | |

⁽¹⁾monoclonal antibodies 8C3
⁽²⁾monoclonal antibodies 6H4

### EXAMPLE 4

The proteins of a serum originating from a healthy individual were separated on a 12% polyacrylamide electrophoresis gel in the presence of sodium dodecyl sulfate (SDS) and then transferred onto a nitrocellulose membrane (Schleicher & Schuell). The membrane was saturated with 2% skimmed milk in PBS (phosphate buffered saline) for 1 hour at ambient temperature and then cut up into several strips. Recombinant bovine protein PrP, identical to that used in example 2, was deposited in the following way onto strips numbered from 1 to 5:

1 μg of recombinant bovine protein PrP, in PBS, was deposited onto strip 1;

1 μg of recombinant bovine protein PrP, in 0.05 mol/l Tris, pH 7.6, 0.15 mol/l NaCl, was deposited onto strips 2 and 5;

1 μg of recombinant bovine protein PrP, in 0.05 mol/l Tris, pH 7.6, 0.15 mol/l NaCl, 0.1 mol/l lysine, was deposited onto strip 4;

1 μg of recombinant bovine protein PrP, in 0.05 mol/l Tris, pH 7.6, 0.15 mol/l NaCl, was deposited onto strip 3, which had been preincubated beforehand for 1 hour with a monoclonal antibody, 8C3, directed against the β2GPI.

After incubation for 1 hour, with stirring and at ambient temperature, strips 1 to 5 were rinsed 4 times with PBS. A solution of monoclonal antibody 6H4, directed against the PrP, diluted 5000-fold in 0.05 mol/l Tris HCl buffer, pH 7.6, 0.15 mol/l NaCl, 0.2% gelatine, 0.05% Tween 20, was added to the strips.

A solution of monoclonal antibody 8C3 directed against the β2GPI, containing 1 μg of antibody/ml in 0.05 ml/l Tris HCl buffer, pH 7.6, 0.15 mol/l NaCl, 0.2% gelatine, 0.05% Tween 20, was added to a strip 6.

After incubation for 1 hour, with stirring and at ambient temperature, strips 1 to 6 were rinsed four times with PBS.

Finally, strips 1 to 6, and also a strip 7 intended to serve as a negative control, were incubated for 1 hour at ambient temperature and with stirring, in the presence of a solution of rabbit antibodies coupled to alkaline phosphatase and directed against mouse antibodies, in a 0.05 mol/l Tris HCl buffer, pH 7.6, 0.2 mol/l NaCl, 0.2% gelatine, 0.05% Tween 20, and then rinsed 6 times with PBS buffer containing 0.05% Tween 20. Revelation of the antibodies was carried out with a liquid mixture of substrate 5-bromo-4-chloro-3-indolyl phosphate/nitro blue tetrazolium (BCIP/NBT). The results obtained are given in FIG. 3.

Strip 6 in FIG. 3 shows the position of the β2 GPI N with a large spot at the level of the monomers and weaker spots at the level of the dimers and polymers.

Strips 1, 2 and 5 show the serum proteins binding the recombinant PrP. These proteins are-located at the same level as those recognized by the monoclonal antibody directed against β2GPI.

Strip 3 shows that the proteins binding the recombinant PrP are those which are recognized by the monoclonal antibody directed against β2GPI (absence of signal after blocking these proteins).

This experiment shows that, among the serum proteins, β2GPI N recognizes and attaches to the recombinant PrP.

1. A method for separating and/or detecting and/or identifying and/or quantifying, in a biological material, at least one prion protein (PrP), characterized in that it comprises a step of separating and/or detecting and/or identifying and/or quantifying a complex (PrP/β2GPI) made up of at least one prion protein bound to at least one form of β2-glycoprotein I (β2GPI).

2. The method as claimed in claim 1, characterized in that it comprises a step of separating and/or detecting and/or identifying and/or quantifying a complex (PrP^{SC}/β2GPI) made up of at least one abnormal prion protein (PrP^{SC}) bound to at least one form of β2GPI, said method constituting a method for separating and/or detecting and/or identifying and/or quantifying, in a biological material, at least one abnormal prion protein.

3. The method as claimed in claim 2, characterized in that, to form a complex (PrP^{SC}/β2GPI), use is made of at least one abnormal prion protein PrP^{SC} originating from scrapie in sheep or goats, bovine encephalopathy, chronic wasting disease in wild ruminants, mink or cat encephalopathies, Creutzfeld-Jacob disease (CJD), Gerstmann-Straussler-Scheinker syndrome (GSS), kuru or fatal familial insomnia.

4. The method as claimed in either of claims 2 and 3, characterized in that, prior to the step of separating and/or detecting and/or identifying and/or quantifying the complex (PrP^{SC}/β2GPI), the biological material is subjected to the action of detergents and/or of enzymes.

5. The method as claimed in claim 4, characterized in that, prior to the step of separating and/or detecting and/or identifying and/or quantifying the complex (PrP^{SC}/β2GPI), the biological material is subjected to the action of proteinase K.

6. The method as claimed in one of claims 1 to 5, characterized in that, to form a complex (PrP/β2GPI), use is made of at least one β2GPI of human origin or of animal origin, a recombinant β2GPI, a β2GPI obtained by chemical synthesis, or a modified form of β2GPI.

7. The method as claimed in one of claims 1 to 6, characterized in that a step of attaching PrP contained in a biological material to at least one form of β2GPI intentionally added to said biological material is carried out so as to form said complex, followed by a step of separating and/or detecting and/or identifying and/or quantifying the complex (PrP/β2GPI).

8. The method as claimed in claim 7, characterized in that the following are carried out:
a step of attaching to a support at least one form of β2GPI or said PrP(s), before or after the step of attaching said PrP(s) to said form(s) of β2GPI so as to form said complex,
a separation step consisting in separating the biological material from the support to which the complex is attached,
a step of detection and/or identification and/or quantification consisting, after said separation step, in detecting and/or identifying and/or quantifying the complex attached to the support, via its component which is not bound to the support.

9. The method as claimed in claim 8, characterized in that a solid support is used as support.

10. The method as claimed in either of claims 8 and 9, characterized in that the attachment to the support is carried out by virtue of a compound which binds to one of the PrP or β2GPI components of the complex, said step of detection and/or identification and/or quantification consisting in detecting and/or identifying and/or quantifying the complex via its component which is not bound to the support.

11. The method as claimed in claim 10, characterized in that the compound which binds to the β2GPI or to the PrP is an antibody which recognizes respectively the β2GPI or the PrP, or else another protein, a biological compound, a chemical compound or a detergent which attaches to the PrP or to the β2GPI.

12. The method as claimed in one of claims 8 to 11, characterized in that:
the step of attaching at least one form of β2GPI or the PrP(s) to a support is carried out by reacting reactive groups of the form(s) of β2GPI or of the PrP(s) with reactive sites of the support, said form(s) being dissolved in a buffer having a pH of between 2.5 and 10.5, preferably between 5.5 and 7.5, so as to obtain a solution having a concentration of between 0.01 and 100 g/l of form(s) of β2GPI or of PrP, the support being kept in contact with the solution at a temperature of between 0° and 40° C. for an incubation period of between 10 seconds and 24 hours, and then
the separation of the support and the solution is carried out by washing the support.

13. The method as claimed in one of claims 7 to 12, characterized in that the step of attaching at least one PrP to at least one form of β2GPI so as to form a complex is carried out by bringing at least one form of β2GPI into contact with the biological material liable to contain PrPs at a temperature of between 00 and 50° C., advantageously in the region of 37° C., for a period of time of between 10 seconds and 24 hours, the biological material being diluted using a buffer giving a pH of between 3.5 and 10, preferably between 5.6 and 7.6.

14. The method as claimed in one of claims 7 to 13, characterized in that the detection and/or identification and/or quantification of the PrP(s) of the complex are carried out using PrP-specific antibodies, or using methods for infecting cells or organisms susceptible to PrP infection.

15. The method as claimed in claim 14, characterized in that the detection and/or identification and/or quantification of the PrP(s) of the complex are carried out using an antibody which specifically recognizes an antigen, preferably protein in nature, of the PrP(s).

16. The method as claimed in one of claims 7 to 13, characterized in that the detection and/or identification and/or quantification of the β2GPI of the complex are carried out using β2GPI-specific antibodies.

17. The method as claimed in one of claims 14 to 16, characterized in that the antibody is coupled to an enzyme label, to colloidal gold, or to a radioactive, fluorescent or luminescent tracer.

18. The method as claimed in claim 17, characterized in that the antibody is coupled to an enzyme label for which the enzyme is brought into contact with a specific substrate able to be converted into a colored product.

19. The method as claimed in one of claims 8 to 15, characterized in that the complex is attached to the support by virtue of its β2GPI component, the separation step comprising isolating the PrP of the complex attached to the support by an affinity chromatography elution method.

20. The method as claimed in claim 19, characterized in that said isolation is carried out by elution of the PrP attached to the solid support using a buffer having a pH of between 2 and 10.5 and an NaCl concentration of between 0 and 5M, preferably using a 0.1 mol/liter glycine-HCl buffer having a pH of 2.5.

21. The method as claimed in one of claims 1 to 6, for separating and/or detecting and/or identifying and/or quantifying at least one PrP in a biological material which naturally contains at least one form of β2GPI, characterized in that it comprises a step of separating and/or detecting and/or identifying and/or quantifying a complex (PrP/β2GPI) made up of at least one PrP bound to at least one form of β2GPI naturally present in said material.
